(19) United States
(12) Patent Application Publication
(10) Pub. No.: US 2019/0247773 A1
(43) Pub. Date: Aug. 15, 2019

(54) MULTILAYER FILTER

(71) Applicant: Aesculap AG, Tuttingen (DE)

(72) Inventor: STEFAN SCHUSTER, Grafenhausen (DE)

(21) Appl. No.: 16/329,315

(22) PCT Filed: Oct. 27, 2017
(86) PCT No.: PCT/EP2017/077647

§ 371 Date: Feb. 28, 2019

(30) Foreign Application Priority Data
Oct. 27, 2016 (DE) 10 2016 120 530.3

(51) Int. Cl. B01D 39/16; B01D 39/18; B32B 1/02; B32B 5/02; B32B 27/12; B32B 27/32; B32B 37/18; B32B 3/04; A61B 50/30; A61L 2/26

(52) U.S. Cl. CPC B01D 39/1623; A61L 2202/24; B32B 1/02; B32B 5/02; B32B 27/12; B32B 27/32; B32B 27/322; B32B 37/185; B32B 3/04; A61B 50/30; A61L 2/26; B01D 2239/065; B01D 2239/10; B01D 2239/1233; B32B 2250/03; B32B 2250/40; B32B 2255/02; B32B 2255/26; B32B 2262/0269; B32B 2307/581; B32B 2307/724; B32B 2323/10; B32B 2377/00; B32B 2439/80; A61B 2050/316; A61L 2202/181; B01D 39/18

(57) ABSTRACT

A multilayer filter of a sterilization container or a sterile packaging includes a core layer made of an aramid fabric, which is covered by or is fully enclosed by at least one outer layer made of at least one other material, preferably PTFE.

### RELATED APPLICATIONS

This application is the United States national phase entry of International Application No. PCT/EP2017/077647, filed Oct. 27, 2017, which claims the benefit of priority of German Application No. 10 2016 120 530.3, filed Oct. 27, 2016. The contents of International Application No. PCT/EP2017/077647 and German Application No. 10 2016 120 530.3 are incorporated by reference herein in their entireties.

### FIELD

The present invention relates to a multilayer filter for a medical sterilization container or a sterile packaging consisting of or equipped with the multilayer filter, a flexible film material for use as a multilayer filter or as a sterile packaging and a method for producing the same. The multilayer filter and the sterile packaging formed therefrom or comprising the same has a core layer made of an aramid fabric which is fully (on both sides) enclosed by at least one outer layer of another material, preferably a filter material, e.g. PTFE according to the sandwich principle (at least three layers).

### BACKGROUND

Sterilization filters (also sterile filters or filter units) are mainly used in medical sterilization containers (also sterile cases or sterile receptacles) for storing surgical instruments, surgical material or other sterile goods. Sterilization filters for the sterile filtration for medical sterilization containers are usually designed in the form of a flat, for instance circular filter disc. The filter disc has fluid exchange openings (also called pores), which enable the exchange of fluids, especially gases, between the environment and the interior of the medical sterilization container. For example, the (medical) sterilization filter is held in a filter holder which is attached to or formed on the medical sterilization container. The filter holder clamps the sterilization filter between a first and a second holding surface or a first and a second holding frame.

Medical sterilization containers are generally used to sterilize, transport and store surgical instruments or material (e.g. artificial implants). To allow superheated steam to enter the medical sterilization container during the sterilization process, preferably in an autoclave, an opening in the container wall is required. However, to prevent germs, bacteria or the like from entering the container through the opening after sterilization, said opening is closed/covered with the aforementioned sterilization filter already before the sterilization process. The sterilization filter allows the fluid exchange but does not allow any germs, bacteria or the like to enter the medical sterilization container. In other words, the opening of the medical sterilization container is closed by a filter unit that has many small fluid exchange openings/pores that allow the exchange of fluids or their molecules but prevent the penetration of germs, bacteria or the like after the sterilization process.

The materials used for sterile filters of sterilization containers or for the sterile packaging of sterile goods are made of cellulose, polypropylene (e.g. polypropylene sold by Kimberly-Clark under the trademark KIMGUARDT), polyethylene (e.g. polyethylene sold by DuPont under the registered trademark TYVEC®) or PTFE (e.g. PTFE sold by DuPont under the registered trademark TEFLON®) according to the generally known state of the art. As a rule, the materials are used as braided fibers, stretched films, or in sintered form for sterile filters or sterile packagings. Polytetrafluorethylene (abbreviation PTFE, occasionally also polytetrafluoroethene) is an unbranched, linearly structured, semi-crystalline polymer of fluorine and carbon. This plastic is often colloquially referred to and sold by DuPont under the registered trademark TEFLON®. Other common PTFE materials include PTFE sold by 3M under the trademark DYNEON™ (formerly HOSTAFLON™) and PTFE sold by W.L. Gore & Associates, Inc. under the registered trademark GORE-TEX® for PTFE diaphragms.

Since sterile filters are used in sterilization containers for the sterilization of surgical instruments or material or as a packaging for sterile goods, it is possible that the sterile filter comes into unintentional contact with surgical instruments and thus a defect can occur in the filter. Surgical instruments, such as knives, scalpels, clamps, clips, needles, saws, cannulas, nails, screwdrivers, sharp spoons, tweezers, scissors, chisels or drills, may have blades and/or tips that can penetrate mechanically into the filter and damage it. A damage or defect occurs if an opening is made in the sterile filter through the filter material which is larger than a fluid exchange opening. A damaged filter cannot therefore guarantee the sterility in the sterilization container after the sterilization process. In other words, the disadvantage of the already known materials is that they offer only low mechanical protection/resistance (e.g. puncture resistance) for the objects packed therein or the instruments stored in the sterilization container. Whenever the filter is damaged, contamination can enter the sterile container.

### SUMMARY

In view of this situation, the object of the present invention is to provide a filter or a sterile packaging that is difficult to damage in the event of an unintentional mechanical contact with the sterile goods, for example surgical instruments or the like. Preferably, a puncture-proof filter or puncture-proof sterile packaging should guarantee a safe, sterile exchange of media in a medical sterilization container or in a packaging for sterile goods, as well as a secure storage of the sterile goods packed therein. The filter/packaging material should preferably be available as a flexible film material. Furthermore, the contents should be protected from contamination that could result from damage to the filter or the like.

This object is achieved by a multilayer, preferably sandwich-type filter for/of a medical sterilization container or a sterile packaging of sterile goods.

The basic idea of the invention is to form the core layer, preferably the middle layer of the (sandwich-like) filter/sterile packaging (material) from an aramid fabric. The core layer is enclosed by at least one outer layer of at least one other (filter) material, particularly in accordance with a sandwich structure (at least three layers), preferably fully enclosed (also on the edge), preferably PTFE. Due to a complete inclusion of the aramid fabric, any aramid fabric fibers that may come loose cannot enter the sterile container.

All layers of the filter, i.e. both the preferably at least two outer layers made of a (filter) material and the core layer made of aramid fabric form/have fluid exchange openings (pores) which enable the exchange of fluids, in particular gases, between the environment and the interior of the sterile container/sterile packaging and provide puncture protection. More specifically, the preferably two outer layers undertake the actual filter function, whereas the aramid fabric (arranged in between) (exclusively) has the puncture protection function, wherein its pores can be (significantly) larger than the pores of the preferably two outer layers. It should be noted here that the filter function of the two outer layers can preferably be achieved due to their respective pore size and/or their respective 3-dimensional (sponge) structure. This means, especially in the latter case, that the pores of the outer layers can be even larger than the smallest germs and yet even the smallest germs cannot pass through the layers.

Thus, only fluids such as air or water vapor can pass through the preferably three-layer membrane formed thereby, while larger particles such as bacteria and spores are retained at least by the outer layers of filter material. If, in the case of a sandwich structure, mechanical action (piercing/cutting) takes place on one of the two outer layers, it may be injured/damaged in such a way that it loses its filter function. However, the filter function of the other outer layer is maintained, since the aramid fabric separating the two outer layers prevents the entire filter/packaging membrane from being pierced/cut through up to the other outer layer and thus at least maintains/protects its filter function.

The fluid exchange openings/pores may have essentially the same size or diameter in all layers of the multilayer filter/sterile packaging, or the fluid exchange openings/pores of the middle puncture protective sheet/protective layer (aramid fabric) may be larger than those of the outer filter/packaging sheets or layers.

The individual sheets/layers may be designed as filter discs in one embodiment. In other words, the filter as seen in cross-section may consist, for example, of at least three layers or at least three individual filter discs. The at least one first, outer (lower) sheet or filter disc may be one of the known filter materials from the group including cellulose or plastic or polymer, preferably polypropylene (e.g. polypropylene sold by Kimberly-Clark under the trademark KIMGUARD™), polyethylene (e.g. polyethylene sold by DuPont under the registered trademark TYVEC®) or PTFE (e.g. PTFE sold by DuPont under the registered trademark TEFLON®), which are available as braided fiber, stretched film or in sintered form. The second layer or filter disc (the middle layer/the core layer) consists of the mentioned aramid fabric. The at least one second, outer (topmost) layer or filter disc lying on top of it also consists of one of the filter materials of the above-mentioned group of the first layer. In this preferred case, the aramid fabric is embedded in the other two materials, so to speak, or in other words it is coated (sandwiched) by them. The two outer layers or filter discs fully enclose the core layer.

At this point it should be explicitly pointed out once again that the term “sandwich-like” requires at least three layers, of which at least one layer is enclosed by two further layers in between. Two-layer composite materials are therefore not “sandwich-like”. Furthermore, the terms sheet, layer and disk are to be understood as a 3-dimensional (sponge) structure which, due to the ratio of the surface to the thickness, bear these designations for reasons of simplicity.

In another embodiment, the core layer made of an aramid fabric may also be directly coated with one of the materials from the group consisting of cellulose or plastic or polymer, in particular with polypropylene and/or PTFE (dip coating, spraying and similar direct coatings), preferably deposited by vaporizing. This structure does not only fully enclose/cover the surface of the core layer, but also the surface of the fluid exchange openings in the core layer is coated (i.e. the filter material does not only cover the two flat sides of the aramid fabric layer facing away from each other but penetrates into their pores). Thus, the entire surface of the aramid fabric, i.e. the core layer, is fully coated with one of the materials mentioned above. This means that the direct coating does not only protect the aramid fabric from UV radiation and chemical influence by acids or similar, but also prevents the shorter fibers of the aramid fabric from separating from the fabric and entering the sterile area. Another advantage of direct coating with the filter material is that the size or diameter of the fluid exchange openings can be controlled directly through the coating time. Such a coating automatically forms fluid exchange openings in the coating that adapt to the fluid exchange openings in the aramid fabric.

Such a multilayer (preferably at least three-layer) filter/sterile packaging ensures a mechanical load-bearing capacity, which is expressed by the fact that the filter/sterile packaging is essentially (more) puncture-proof and cannot be punctured or damaged or hardly punctured or damaged by a sharp or pointed surgical instrument. On the other hand, the multilayered (preferably at least three-layer) structure also prevents aramid fibers from being released from the aramid fabric and entering the sterile space. At the same time, the filter/sterile packaging consisting of several layers remains elastic and insensitive to compressive and bending stresses. A multilayer filter/sterile packaging as described above allows longer maintenance intervals, which means that replacement is rarely necessary, which in turn leads to a reduction in costs. Another advantage of the already mentioned filter materials from the group including cellulose or plastics, especially polypropylene or PTFE, is that these materials protect the aramid fabric from UV radiation. In aramid fabrics, UV radiation initially causes a visible discoloration from the original light yellow to a bronze-brown hue. After prolonged exposure to UV radiation, the fiber loses up to 75% of its strength.

In other embodiments, the filter/sterile packaging may also have more than three layers as seen in cross-section, for example several aramid layers or several layers of the known filter materials, preferably plastic, which are arranged between the aramid layers and/or also envelope the at least one aramid layer. In a yet other embodiment, the multilayer filter/sterile packaging may also be formed from only two layers or filter discs. In an embodiment with two filter discs, the aramid layer is on the side facing away from the sterilization goods and the other filter layer is on the side facing the sterilization goods.

Aromatic polyamide (aramid) is sold by DuPont under the registered trademark KEVLAR®. Aramid fibers have a high specific (weight-related) strength, low density, high impact strength, good heat resistance and dimensional stability, good vibration damping and a high energy absorption capacity. Aramid fibers have good resistance to solvents, fuels, lubricants, salt water, etc.; however, aramid fibers are attacked by some strong acids and alkaline solutions. They are resistant to attacks by fungi and bacteria. Aramid fibers are widely used. They are mainly known for their use in safety clothing (splinter vests, cut-resistant gloves). The fibers have a high mechanical strength.

Since the individual aramid fibers are short, they may become detached from the aramid fabric. To prevent these fibers from entering the medical sterilization container or the sterilized area of a sterile packaging, the aramid fabric is enclosed by another filter material made of one of the already known materials, which means that the filter has several layers, i.e. in this case a sandwich-like structure. In the preferred exemplary embodiment, the filter/sterile packaging is constructed in three sheets, with the middle sheet consisting of the aramid fabric which is enclosed on both sides by a filter material from the above-mentioned known group, preferably PTFE. The aramid fabric in the multilayer filter/sterile packaging comprising an aramid core is thus flexible, if necessary elastic and/or insensitive to compressive and bending stresses.

The filter/sterile packaging, more precisely, the preferably at least two outer layers or filter discs, may have a larger surface diameter than the core layer in an exemplary embodiment and be glued/bonded together at their outer edges of the discs, or the outer layers can also be glued directly to the core layer. Such an assembly is easy to realize, which lowers the production costs. Due to this structure, the core layer is preferably fully enclosed by the two outer layers.

The core layer and the outer layers of the filter/sterile packaging have fluid exchange openings/pores, as already explained above. In one embodiment, the fluid exchange openings of the core layer may preferably have larger diameters than the fluid exchange openings of the outer layer. This has the advantage that a coarser and therefore cheaper puncture-resistant aramid fabric can be used in the core, and a finer material, i.e. a material with smaller fluid exchange openings, can be used for the outer layer.

The fluid exchange openings of the at least one outer layer may be smaller than the diameter of fibers of the aramid fabric in one embodiment. The fact that the fibers cannot pass through the outer layer ensures that the fibers do not enter the sterilized space and contaminate it.

The multilayer filter may be essentially circular, square or rectangular. It can also have any other shape to fit a filter holding device in a medical sterilization container. In addition, other shapes are also possible, such as bags or pouches made of this multilayer filter composite material for packaging surgical instruments or material. Although the filter is preferably a permanent filter, it may also be designed as a disposable filter.

Furthermore, a process for producing a multilayer filter for/of a medical sterilization container or multilayer sterile packaging will be presented. The process comprises enclosing a core layer of aramid fabric with at least one other (filter) material, wherein the at least one other material is selected from the group including cellulose or plastic, preferably is polypropylene (e.g. polypropylene sold by Kimberly-Clark under the trademark KIMGUARD™), polyethylene (e.g. polyethylene sold by DuPont under the registered trademark TYVEC®) or PTFE (e.g. PTFE sold by DuPont under the registered trademark TEFLON®), or another (filter) material with similar/comparable mechanical/chemical properties.

The process of manufacturing the filter may also include to glue/bond two separate filter discs or outer filter layers to each other. The filter discs/layers are preferably made of a material from the group including cellulose or plastic, preferably of (e.g. polypropylene sold by Kimberly-Clark under the trademark KIMGUARD™), polyethylene (e.g. polyethylene sold by DuPont under the registered trademark TYVEC®) or PTFE (e.g. PTFE sold by DuPont under the registered trademark TEFLON®). The filter discs/layers (enclosing the aramid fabric) are glued to each other at their outer edge. The outer edge is to be understood as the (frame) surface which is on that side which faces the other filter disc/filter layer and is close to the outer circumference of the filter disc/filter layer, while the proximity to the outer circumference is to be understood preferably as a distance to the outer circumference of the respective filter disc/filter layer which is less than ninety percent of the radius/cross-sectional dimension, preferably less than 50 percent and most preferably less than 10 percent of the radius/cross-sectional dimension.

Another process of manufacturing involves enclosing the core layer of aramid fabric by (direct) coating, preferably by vapor deposition, of the core layer, particularly preferred with a material of the group including cellulose or plastic, preferably with polypropylene (e.g. polypropylene sold by Kimberly-Clark under the trademark KIMGUARD™), polyethylene (e.g. polyethylene sold by DuPont under the registered trademark TYVEC®) or PTFE (e.g. PTFE sold by DuPont under the registered trademark TEFLON®), whereby the (filter) material is applied directly to the aramid fabric in particular by vapor deposition. Thus, each surface area of the intermediate layer (aramid fabric) is fully coated with the (filter) material.

In a preferred embodiment, a medical sterile packaging has a multilayer filter consisting of a core layer or core sheet of aramid fabric as puncture protection. The core layer or core sheet is fully enclosed by at least one first outer layer or outer sheet made of a filter material on one side thereof and/or by at least one second outer layer or outer sheet made of a filter material on the other side thereof (beyond the peripheral edge of the core layer).

### BRIEF DESCRIPTION OF THE DRAWING FIGURES

The present invention will be explained in more detail below on the basis of preferred embodiments with reference to the accompanying Figures.

FIG. 1 shows a medical sterilization container.

FIG. 2 shows a filter holding device for a medical sterilization container.

FIG. 3 shows the process of manufacturing a first embodiment of a multilayer filter.

FIG. 4 shows the structure of the first embodiment of a multilayer filter in a cross-sectional view.

FIG. 5 shows the structure of a second embodiment of a multilayer filter in a cross-sectional view.

FIG. 6 shows the process of manufacturing a third embodiment of a multilayer filter.

FIG. 7 shows the structure of a third embodiment of a multilayer filter in a cross-sectional view.

### DETAILED DESCRIPTION

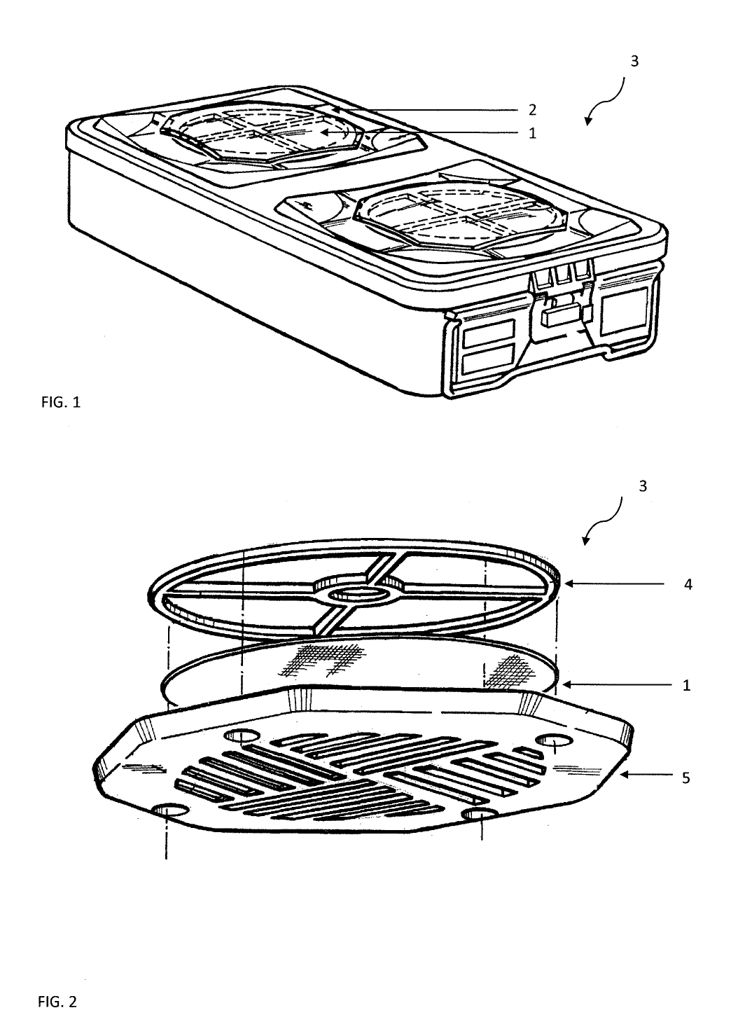

FIG. 1 illustrates a multilayer filter 1 in a holding device 2 of a medical sterilization container 3. The filter 1 is designed as an approximately circular disc and is enclosed in the frame-like holding device 2, preferably clamped therein, which in turn is attached to the medical sterilization container 3.

FIG. 2 illustrates the structure of the filter holding device 2 for the multilayer filter 1 in a medical sterilization container 3. The filter holding device 3 consists of a truss-shaped holding ring 4 and a holding plate 5 in the manner of a grating or perforated plate, which hold/clamp the multilayer filter 1 between them. As a result, openings have been created in the filter holding device 3, both in the holding ring 4 and in the holding plate 5, which allow a fluid exchange between the interior of the sterilization container 3 and its surroundings and simultaneously protect the multilayer filter 1 from intense/large-area force influences.

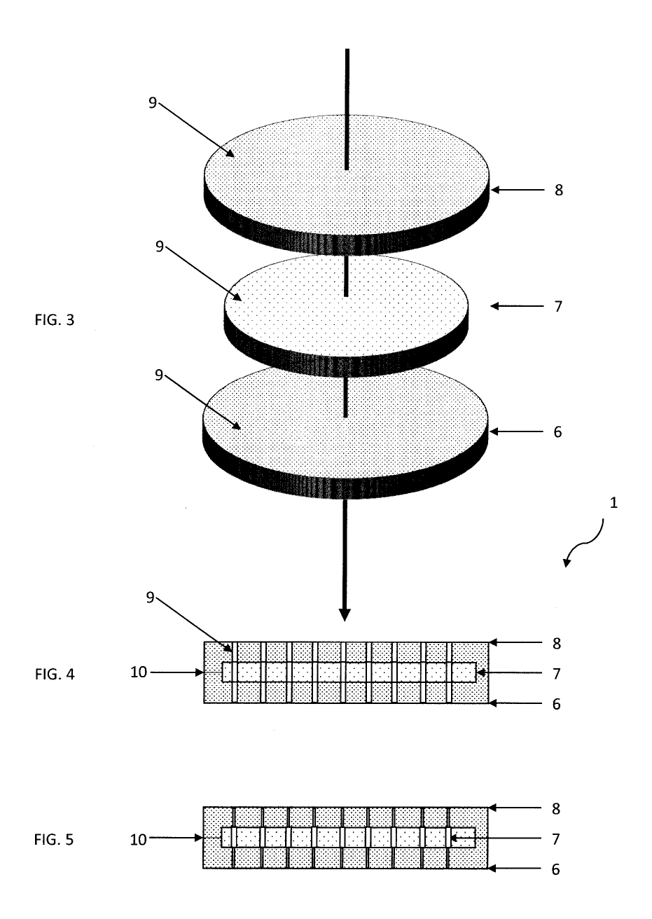

FIG. 3 shows the manufacturing process and the constructional assembly of the multilayer filter 1 according to a first embodiment of the present invention. Accordingly, all elements of the multilayer filter 1 (composite material/composite membrane) are stacked one on top of the other, so that they are all arranged centrally along an imaginary axis (in the membrane thickness direction). The term “elements” are to be understood as the individual layers or filter discs of the multilayer filter 1.

The multilayer filter 1 has a lowermost/outermost first layer/sheet 6 made of a filter material preferably from the group consisting of cellulose or plastic, preferably made of polypropylene (e.g. polypropylene sold by Kimberly-Clark under the trademark KIMGUARD™), polyethylene (e.g. polyethylene sold by DuPont under the registered trademark TYVEC®) or PTFE (e.g. PTFE sold by DuPont under the registered trademark TEFLON®). The at least one middle, second layer/sheet, namely the core layer 7, consists of an aramid fabric. The uppermost/outermost third layer 8 is again made of a material from the group including cellulose or plastic, preferably of polypropylene (e.g. polypropylene sold by Kimberly-Clark under the trademark KIMGUARD™), polyethylene (e.g. polyethylene sold by DuPont under the registered trademark TYVEC®) or PTFE (e.g. PTFE sold by DuPont under the registered trademark TEFLON®). This preferred sandwich-like structure is exemplary and may also have several other layers/sheets of aramid as well as several layers/sheets of the above-mentioned filter materials or materials with similar (filter) properties. All layers/sheets have fluid exchange openings (pores) 9, with the first and third layer/sheet 6, 8 having fluid exchange opening diameters (pore size) which are smaller than the fiber diameter of the aramid fabric of the second layer 7.

In the manufacturing process for the first embodiment of the multilayer filter 1, the individual layers/sheets are bonded by means of an adhesive or by ultrasonic welding near the outer circumference of the two outer layers/sheets 6, 8 and thus (loosely) enclose the second layer 7, namely the aramid fabric between them.

The two outer layers/sheets made of a suitable filter material thus form the actual filter membrane with corresponding filter properties, whereas the middle layer/sheet made of the aramid fabric (exclusively) represents a puncture protection. If the filter 1 is damaged on one flat side by the action of a mechanical force and thus the filter property of the one outer layer/sheet is destroyed/impaired, the intermediate layer of aramid fabric leaves the other outer layer/sheet undamaged and thus its filter effect intact.

FIG. 4 shows the cross-section of the first embodiment of the multilayer filter 1 according to the first embodiment. In this embodiment, all fluid exchange openings or pores 9 of the layers have the same fluid exchange opening diameter/pore diameter. The intermediate layer of aramid fabric, i.e. the second layer 7, is embedded between the two other, outer layers/sheets 6 and 8 and fully enclosed. The peripheral edge-side contact/connection areas 10 of the two outer layers/sheets are bonded together, but can also be braided, caulked, riveted or similar due to production. In this embodiment, only the two outer layers/sheets are firmly connected to each other and the middle layer/sheet is loosely inserted between them. The two outer layers/sheets may also be firmly bonded (glued) to the central filter. Other exemplary embodiments of bonding are also obvious, which include a complete enclosure of the core layer, e.g. welding (preferably ultrasonic welding) or pressing.

FIG. 5 shows a second embodiment of the multilayer filter 1 in which the two outer layers/sheets have a fluid exchange opening diameter or pore diameter which is smaller than the fluid exchange opening diameter of the core layer 7. The method for its manufacture corresponds to the first preferred exemplary embodiment.

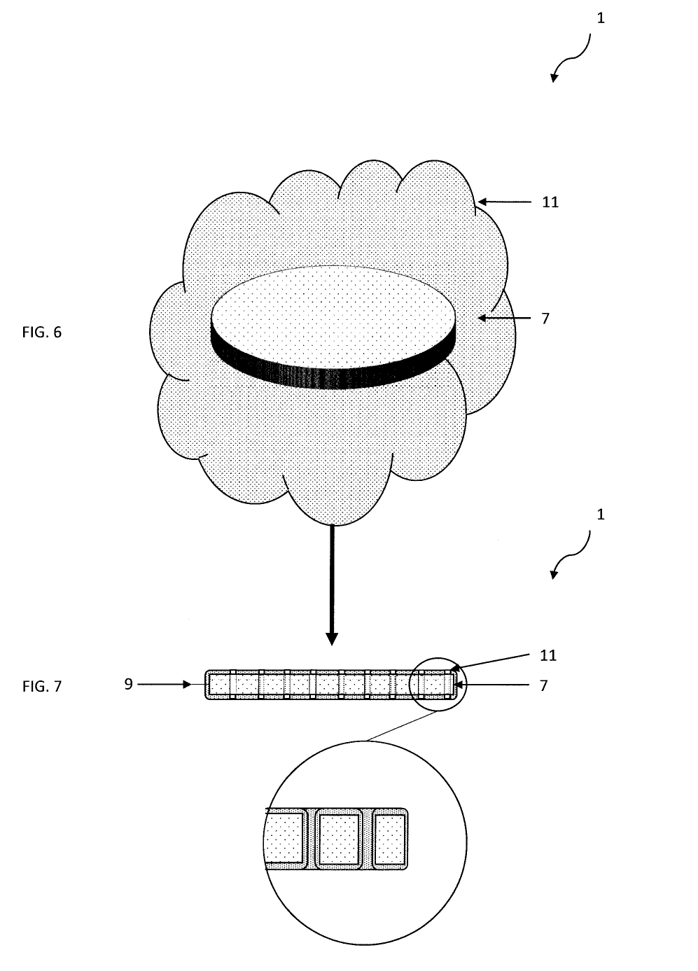

FIG. 6 shows the process of manufacturing a third embodiment of the multilayer filter 1. Here, the aramid core, i.e. the middle layer, is coated with a coating material 11 from the group consisting of cellulose or plastic, preferably of a polymer, especially polypropylene (e.g. polypropylene sold by Kimberly-Clark under the trademark KIMGUARD™), polyethylene (e.g. polyethylene sold by DuPont under the registered trademark TYVEC®) or PTFE (e.g. PTFE sold by DuPont under the registered trademark TEFLON®). This can be done by plastic and powder coating, wet painting, a spray-sintering method, electrothermal processes and evaporation.

FIG. 7 shows the cross-section of a third embodiment of the multilayer filter 1 as a whole and in detail. In this embodiment, all outer sides (top and bottom) of the aramid fabric 7 and also all fluid exchange openings/pores 9 (inside the pores) formed by the aramid fabric 7 are coated with a (filter) material 11 of the already known group. The coating not only reaches the layer surfaces or disc surfaces of the aramid fabric 7 but also the inner surfaces of the fluid exchange openings/pores 9. The multilayer filter 1 according to the third preferred exemplary embodiment of the present invention is not formed from three separate layers in this embodiment but from a middle layer 7, namely the aramid fabric, and a coating 11 which fully envelops the aramid fabric and penetrates its pores.

In summary, the invention relates to a multilayer filter of a (medical) sterile container or a (medical) sterile packaging each consisting of a core layer of an aramid fabric, which is covered or fully enclosed by at least one outer layer of at least one other material, preferably PTFE and most preferably PTFE sold by DuPont under the registered trademark TEFLON®, a method for producing the multilayer (medical) filter of a sterile container or for producing the (medical) sterile packaging, and the use of a composite membrane as a multilayer filter of a (medical) sterile container or as a (medical) sterile packaging, the composite membrane consisting of a core layer made of an aramid fabric, which is covered or fully enclosed by at least one outer layer made of at least one other material, preferably PTFE and most preferably PTFE sold by DuPont under the registered trademark TEFLON®.

1. A multilayer material for a medical sterilization container or for sterile packaging, the multilayer material comprising a core layer made of an aramid fabric as puncture protection, which is fully enclosed by at least one first outer layer made of filter material on one side thereof and by at least one second outer layer made of filter material on another side thereof.

2. The multilayer material according to claim 1, wherein the core layer, the at least one first outer layer and the at least one second outer layer have fluid exchange openings or pores which allow an exchange of fluids between an exterior and an interior of the medical sterilization container or sterile packaging.

3. The multilayer material according to claim 2, wherein the at least one first outer layer and/or the at least one second outer layer comprises cellulose or plastic.

4. The multilayer material according to claim 3, wherein the at least one first outer layer and the at least one second outer layer either:
have a larger surface diameter than the core layer and are bonded together at their outer edge surrounding the core layer;
are directly bonded to the core layer.

5. The multilayer material according to claim 4, wherein the aramid fabric comprises fibers each having a diameter, and the fluid exchange openings or pores of the at least one first outer layer and/or the at least one second outer layer are smaller than the diameter of fibers of the aramid fabric.

6. The multilayer material according to claim 3, wherein the core layer is coated with a coating material that comprises cellulose, polymer, polypropylene and/or PTFE.

7. The multilayer material according to claim 3, wherein the fluid exchange openings or pores of the core layer have larger diameters than the fluid exchange openings or pores of the at least one first outer layer and of the at least one second outer layer.

8. A method for producing a multilayer filter of a medical sterilization container or for producing a medical sterile packaging, comprising the following method steps:
providing a core layer made of aramid fabric as puncture protection, the core layer comprising an inner side and an outer side,
covering the core layer on the inner side with at least one first outer layer made of filter material, and
covering the core layer on the outer side with at least one second outer layer made of filter material, so that the core layer is fully enclosed.

9. The method according to claim 8, wherein, the at least one first outer layer and the at least one second outer layer are formed from two separate filter discs or membranes, which are directly bonded together at their respective outer edge so as to enclose the core layer.

10. The method according to claim 8, wherein the core layer is coated with the at least one first outer layer and the at least one second outer layer.

11. A medical sterilization container comprising a multilayer material according to claim 1.

12. A medical sterile packaging comprising a multilayer material according to claim 1.
